(19) United States
(12) Patent Application Publication
(10) Pub. No.: US 2005/0136097 A1
(43) Pub. Date: Jun. 23, 2005

(54) Soft paper-based products

(75) Inventor: Jay C. Hsu, Alpharetta, GA (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/742,623

(22) Filed: Dec. 19, 2003

(51) Int. Cl. A61K 9/70

(52) U.S. Cl. 424/443; 162/164.4

(57) ABSTRACT

The present invention is generally related to a soft paper-based product comprising a cellulosic fibrous material, wherein an aqueous-based softening composition is incorporated into the paper-based product. The softening composition includes a silicone glycol, a silicone quaternary ammonium compound, an emollient, and water. The softening composition may be incorporated into a paper-based product such that the add-on level of the softening composition is between about 0.1% to about 10% by weight of the paper-based product. In some embodiments, the softening composition is an oil-in-water emulsion. The present invention is also directed to a method for forming soft paper products, where a cellulosic fibrous web is treated with an aqueous-based softening composition, which includes a silicone glycol, a silicone quaternary ammonium compound, an emollient, and water.

### BACKGROUND OF THE INVENTION

Consumers use paper-based products, such as paper towels, tissues, and paper-based wipers for a wide variety of applications. For example, various types of paper-based products are used for personal care, nose care, cosmetic applications, eyeglass cleaning, and so forth. Efforts are continually being made to improve various properties of such paper-based products in order to provide better products to the consumer. One of the properties constantly sought to be improved is softness.

Previous methods for imparting softness to paper products are known in the art. For example, U.S. Pat. No. 6,428,794 to Klofta, et al. describes a lotion composition applied to tissue paper which is semi-solid at ambient temperature and which comprises a substantially water free petrolatum emollient, an immobilizing agent for immobilizing the petrolatum emollient on the surface of the treated tissue paper, a nonionic hydrophilic surfactant (such as a C₈-C₂₂ ethoxylated alcohol or an ethoxylated sorbitan ester of a C₁₂-C₁₈ fatty acid), and an additional hydrophilic surfactant, which may include a silicone polyether copolymer.

Despite various methods known for imparting softness to paper-based products, there is a continuing need for paper-based products having improved softness. Particularly, there is a continuing need for methods for making paper-based products softer that do not significantly decrease the absorbency or the strength of the paper-based products.

### SUMMARY OF THE INVENTION

In accordance with one embodiment of the present invention, a soft paper-based product is disclosed that comprises a cellulosic fibrous material. An aqueous-based softening composition is incorporated into the paper-based product at an add-on level of between about 0.1% to about 10% by weight of the paper-based product. The softening composition comprises a silicone glycol in an amount between about 0.01% to about 20% by weight of said softening composition, a silicone quaternary ammonium compound in an amount between about 0.01% to about 20% by weight of said softening composition, an emollient in an amount between about 0.01% to about 20% by weight of said softening composition, and water in an amount greater than about 40% by weight of said softening composition.

In accordance with another embodiment of the present invention, a method for forming a soft paper product is disclosed. The method comprises forming a web from at least one furnish containing cellulosic fibrous material and water. The web is treated with an aqueous-based softening composition such that the add-on level of the softening composition is between about 0.1% to about 10% by weight of the paper product. The softening composition comprises a silicone glycol in an amount between about 0.01% to about 20% by weight of the softening composition, a silicone quaternary ammonium compound in an amount between about 0.01% to about 20% by weight of the softening composition, an emollient in an amount between about 0.01% to about 20% by weight of the softening composition, and water in an amount greater than about 40% by weight of the softening composition.

In accordance with an additional embodiment of the present invention, an aqueous-based softening composition is disclosed. The softening composition comprises a silicone glycol in an amount between about 0.01% to about 20% by weight of the softening composition, a silicone quaternary ammonium compound in an amount between about 0.01% to about 20% by weight of the softening composition, an emollient in an amount between about 0.01% to about 20% by weight of the softening composition, and water in an amount greater than about 40% by weight of the softening composition.

Other features and aspects of the present invention are discussed in greater detail below.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment may be used in another embodiment to yield a still further embodiment. Thus, it is intended that the present invention cover such modifications and variations as come within the scope of the appended claims and their equivalents.

In general, the present invention is directed to a paper-based product into which a softening composition is incorporated, such that the paper-based product has improved softness. The softening composition is an aqueous-based composition which includes a silicone glycol, a silicone quaternary ammonium compound, an emollient, and water. It has been unexpectedly discovered that a softening composition made from the particular ingredients disclosed herein, and the respective amounts of those ingredients, may provide a synergistic effect in terms of improved softness when incorporated into a paper-based product. Moreover, it has been unexpectedly discovered that the softening composition used in the present invention may be incorporated at relatively low add-on levels to a paper-based product and still impart improved softness to the paper-based product without significantly decreasing the strength or the absorbency of the treated paper-based product.

As stated above, the softening composition according to the present invention is an aqueous-based composition. Water is typically present within the softening composition in an amount greater than about 40% by weight of the softening composition, and in some embodiments, greater than about 75% by weight of the softening composition.

The aqueous-based softening composition according to the present invention includes a silicone glycol. In particular, the silicone glycol may be a nonionic dimethicone copolyol having the following general formula: 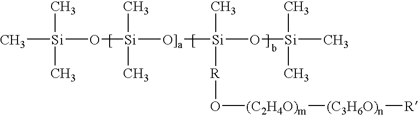
wherein a≧1 and b≧1;
wherein R is selected from C₁-C₆ alkyl groups and C₁-C₆ hydroxyalkyl groups. In general, the C₁-C₆ alkyls and/or C₁-C₆ hydroxyalkyls may be linear or branched. Moreover, although not required, in some embodiments, the C₁-C₆ alkyls and/or C₁-C₆ hydroxyalkyls may be substituted with any of a variety of substituents;
wherein R′ is selected from hydrogen, C₁-C₆ alkyl groups, C₁-C₆ hydroxyalkyl groups, C₁-C₆ acyl groups, and C₁-C₆ acetate groups. In general, R′ may be linear or branched. Moreover, although not required, in some embodiments, R′ may be substituted with any of a variety of substituents;
wherein m is between about 1 to about 500; and
wherein n is between about 1 to about 300.

The “n/m ratio” may be adjusted to control the water absorption characteristics or hydrophilicity of the silicone glycol. As used herein, the term “n/m ratio” refers generally to the ratio of propyoxylate groups (i.e., —C₃H₆O—) to ethyoxylate groups (i.e., —C₂H₄O—) in a dimethicone copolyol as shown in the above formula. In particular, by decreasing the n/m ratio, the resulting dimethicone copolyol may become more hydrophilic (i.e., may have a greater affinity for water). In turn, a softening composition that is incorporated with a relatively hydrophilic dimethicone copolyol may be more compatible with an aqueous environment. As a result, because the composition has some affinity for water, it may generally be incorporated into a paper-based product without causing a substantial decrease in the absorbency (i.e., hydrophilic) properties of the paper-based product.

In addition, the molecular weight of the silicone glycol utilized in. the softening composition of the present invention may also vary. For instance, dimethicone copolyols having a lower molecular weight (e.g., less than about 8,000) tend to feel slippery or silky. The molecular weight may affect other properties as well, such as intrinsic viscosity, emulsifying capability, and so forth. Thus, to achieve a paper-based product having a particularly desired hand feel, the molecular weight of the silicone glycol may be selectively varied. Moreover, besides, or in conjunction with varying the molecular weight of the silicone glycol, the n/m ratio may also be varied, as stated above, to further assist the attainment of a particular hand feel. Further, in some embodiments, the silicone glycol may reduce the surface tension of the softening composition, thereby rendering the softening composition more stable.

Some specific examples of suitable silicone glycols include Abil B8852, Abil B8863, Abil B88183, and Abil B8843, which are commercially available from Goldschmidt Chemical Corp. in Dublin, Ohio. Additional silicone glycols which may be suitable for the softening composition of the present invention include Dow Corning 190, commercially available from Dow Corning Corporation. Other suitable silicone glycols are discussed, for example, in U.S. Pat. No. 5,552,020 to Smith, et al. and U.S. Pat. No. 6,504,412 to Schroeder, which are incorporated herein in their entirety by reference thereto for all purposes.

The amount of the silicone glycol in the softening composition may generally vary. For example, in some embodiments, the amount of the silicone glycol may be between about 0.01% to about 20% by weight of the softening composition, in some embodiments between about 0.01% to about 10% by weight of the softening composition, and in some embodiments, between about 0.01% to about 5% by weight of the softening composition.

Besides the silicone glycol described above, the aqueous-based softening composition of the present invention also includes a silicone quaternary ammonium compound. In certain embodiments, the silicone quaternary compound may enhance the lubricating, conditioning, or softening effects of the composition on the paper-based product. In some embodiments, the silicone quaternary ammonium compound used herein is cationic so as to aid in the attachment or bonding of the softening composition to the cellulosic fibers of a paper-based product, which are anionic in nature. Moreover, a cationic silicone quaternary ammonium compound may also enhance the ability of the cellulosic fibers of the paper-based product to retain other non-cationic components, such as the nonionic silicone glycol component; discussed in greater detail above. Further, the silicone quaternary ammonium compound may have antistatic properties to reduce static build-up, and the silicone quaternary ammonium compound may also aid in improving the water solubility and/or dispersibility of the paper-based product.

In some instances, the silicone quaternary ammonium compound used in the present invention has one or more amino groups that are linked together by various elements. For example, diquaternary and polyquaternary ammonium compounds may be used as the silicone quaternary ammonium compound. Particular examples of silicone quaternary ammonium compounds that may be used in the softening composition of the present invention are silicone diquaternary ammonium salts. For instance, in one embodiment of the present invention, a silicone diquaternary ammonium salt is used which has the following general formula: 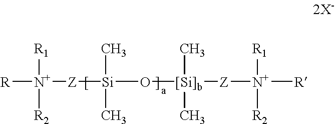
wherein a≧1 and b≧1;
wherein R₁ and R₂ are independently selected from C₁-C₆ alkyl groups and C₁-C₆ hydroxyalkyl groups. In general, the C₁-C₆ alkyls and/or C₁-C₆ hydroxyalkyls may be linear or branched. Moreover, although not required, in some embodiments, the C₁-C₆ alkyls and/or C₁-C₆ hydroxyalkyls may be substituted with any of a variety of substituents;
wherein R and R′ are independently selected from C₈-C₂₄ aliphatic hydrocarbons. In general, the C₈-C₂₄ aliphatic hydrocarbons may be linear or branched. Moreover, although not required, in some embodiments, the C₈-C₂₄ aliphatic hydrocarbons may be substituted with any of a variety of substituents;
wherein Z is —(CH₂)ⱼ—CHOH—CH₂—O—(CH₂)ₖ—, wherein j≧1 and k≧1; or wherein Z is CH₂—CH₂—CH₂—O—(CH₂)₃—; or wherein Z is an alkyl group or an alkyl ester; and
wherein X may be selected from any suitable or compatible counterion, including, for example, alkoxy, chloride, methylsulfate, ethylsulfate, acetate, lactate, and so forth.

Suitable silicone quaternary ammonium compounds are discussed, for example, in Smith, et al. and Schroeder, noted above. For instance, one specific example of a silicone diquaternary ammonium compound that may be employed in the softening composition of the present invention is Albiquat 3272, commercially available from Goldschmidt Corp.

The amount of the silicone quaternary ammonium compound in the softening composition may generally vary. For example, in some embodiments, the amount of the silicone quaternary ammonium compound may be between about 0.01% to about 20% by weight of the softening composition, in some embodiments between about 0.01% to about 10% by weight of the softening composition, and in some embodiments, between about 0.01% to about 5% by weight of the softening composition.

Besides the above-described ingredients, the aqueous-based softening composition used for softening paper-based products according to the present invention also includes one or more emollients. Generally, an emollient imparts a soft, lubricious, lotion-like feel to the paper-based product into which the softening composition is incorporated. Additionally, the emollient may lubricate or soothe the skin of a user of the chemically treated paper-based product.

Some emollients that may be suitable for the present softening composition include, but are not limited to, petroleum or mineral oils, such as petrolatum; animal oils, such as mink oil and lanolin oil; plant oils, such as sunflower oil, soy sterol, and avocado oil; silicone oils, such as alkyl silicones; and so forth. Additional suitable emollients may include those discussed in International Patent Publication No. WO 02/41869, published on May 30, 2002, which is incorporated herein in its entirety by reference thereto for all purposes. Other suitable emollients may include PEG 75 lanolin, capric acid, caproic acid, caprylic acid, caprylicicapric mixed acids, caprylic/capric triglyceride (e.g., Crodamol GTCC sold by Croda, Inc.), cholesterol, lauric acid, magnesium stearate, myristic acid, oleic acid, palmitic acid, pentaerythritol, sorbitol, stearic acid, stearols (vegetable), methyl gluceth 20 benzoate, linear primary alkyl esters of benzoic acid (e.g., C₁₂-C₁₅ alkyl benzoate), ethoxylated cetyl stearyl alcohol, Finsolv® SLB 101 or SLB 201 (sold by Finetex Corp.). Still other suitable emollients are described in U.S. Pat. No. 4,559,157 to Smith et al., U.S. Pat. No. 4,690,821 to Smith et al., U.S. Pat. No. 5,830,487 to Klofta, et al., and U.S. Pat. No. 5,871,763 to Luu, et al., which are incorporated herein in their entirety by reference thereto for all purposes:

The amount of the emollient in the softening composition may generally vary. For example, in some embodiments, the amount of the emollient may be between about 0.01% to about 20% by weight of the softening composition, in some embodiments between about 0.01% to about 10% by weight of the softening composition, and in some embodiments, between about 0.01% to about 5% by weight of the softening composition.

Besides the above-identified ingredients, the aqueous-based softening composition used in the present invention to treat paper-based products may also contain, in certain embodiments, one or more fatty alcohols. Fatty alcohols may prevent the softening composition from substantially migrating into the interior of the paper-based product when incorporated at the surface of a paper-based product, which may allow for lower add-on levels of softening composition. Fatty alcohols suitable for the softening composition used in the present invention may include, but are not limited to, alcohols having a carbon chain length of C₁₄-C₃₀, including, for example, cetyl alcohol, stearyl alcohol, cetearyl alcohol (which is a mixture of cetyl alcohol and stearyl alcohols), arachidyl alcohol, and behenyl alcohol. Other suitable fatty alcohols are described in U.S. Pat. No. 5,830,487 to Klofta, et al. and U.S. Pat. No. 5,871,763 to Luu, et al. In some embodiments, for instance, three fatty alcohols may be utilized in the softening composition for treating paper-based products. For example, the combination of cetyl alcohol, stearyl alcohol, and cetearyl alcohol may be utilized in the softening composition for treating paper-based products.

In embodiments where a fatty alcohol is present in the softening composition, the amount of fatty alcohol may vary, depending on the amount of the emollient utilized. For instance, in some embodiments, the amount of fatty alcohol in the softening composition may be between about 0.01% to about 20% by weight of the softening composition, in some embodiments between about 0.01% to about 10% by weight of the softening composition, and in some embodiments, between about 0.01% to about 5% by weight of the softening composition.

Besides the above-described ingredients, the aqueous-based softening composition used in the present invention to soften paper-based products may include, in certain embodiments, one or more skin conditioning agents. A skin conditioning agent generally refers to any material that may enhance certain properties of the skin, such as moisturize the skin, soften the skin, protect the skin, and so forth. For example, in one embodiment, the skin conditioning agent may include a humectant (i.e., a compound that has an affinity for water). In general, a variety of humectants may be suitable for use in the softening composition of the present invention. Some examples of suitable humectants include, but are not limited to, glycerin; ethoxylated glycerins, such as POE-26 glycerin, POE-7 glycerin, sorbitol, 1,2,6-hexanetriol sorbitol, and hydroxypropyl sorbitol; phosphinic carboxylic acid (PCA) and salts thereof, such as sodium PCA; alpha hydroxy acids and salts thereof, such as lactic acid, sodium lactate, and glycolic acid; glucose derivatives, such as glucose glutamate; polyalkylene glycols and alkylene polyols and their derivatives, including propylene glycol, dipropylene glycol, polypropylene glycol, polyethylene glycol, 1,3-butylene glycol, triethylene glycol, and dipropylene glycol; and other humectants, such as maltodextrin, maltitol, mannitol, zylitol, sodium polyaspartate, ethoxylated castor oil, various humectants available from Lipo Chemicals (e.g., acetamide MEA, ethoxylated glycerin, and lactamide MEA), and so forth.

Still other skin conditioning agents may also be utilized in the softening composition of the present invention. For instance, other skin conditioning agents that may be suitable for use include, but are not limited to, dimethicone, glyceryl stearate, caprylic/capric stearate triglyceride, stearamidopropyl PG-dimonium chloride phosphate and cetyl alcohol (i.e., phospholipid SV), and so forth. Still other suitable skin conditioning agents are described in U.S. Pat. No. 4,559,157 to Smith et al., U.S. Pat. No. 4,690,821 to Smith et al., U.S. Pat. No. 5,830,487 to Klofta, et al., and U.S. Pat. No. 5,871,763 to Luu. et al., which are incorporated herein in their entirety by reference thereto for all purposes.

In embodiments where skin conditioning agent(s) are present, the amount of skin conditioning agent may vary. For instance, in certain embodiments, the amount of skin conditioning agent may be between about 0.01% to about 20% by weight of the softening composition, in some embodiments between about 0.01% to about 10% by weight of the softening composition, and in some embodiments, between about 0.01% to about 5% by weight of the softening composition.

In one particular embodiment, several skin conditioning agents are used in the softening composition, including glycerin, dimethicone, caprylic/capric stearate triglyceride, and stearamidopropyl PG-dimonium chloride phosphate and cetyl alcohol. For example, in such an embodiment, the amount of glycerin, dimethicone, caprylic/capric stearate triglyceride, and stearamidopropyl PG-dimonium chloride phosphate and cetyl alcohol, may each be between about 0.01% to about 20% by weight of the softening composition, in some embodiments between about 0.01% to about 10% by weight of the softening composition, and in some embodiments, between about 0.01% to about 5% by weight of the softening composition. It should also be understood that some of the ingredients mentioned above (e.g., emollient, fatty alcohol, etc.) and/or other ingredients may also act as skin conditioning agents as well.

In certain embodiments, the aqueous-based softening composition used in the present invention may be an oil-in-water emulsion. As used herein, the term “oil-in-water emulsion” generally refers to a stable dispersion or suspension of finely divided liquid droplets (the oil phase or the discontinuous phase) in a second liquid (the water phase or the continuous phase). An oil-in-water emulsion typically comprises an emulsifier, which aids in dispersing the oil phase into the water phase of the softening composition. In embodiments where an emulsifier is present in the softening composition, any of a variety of emulsifiers may be utilized, including, for example, nonionic, anionic, amphoteric, and/or cationic emulsifiers. Typically, the emulsifier or blend of emulsifiers may have an overall hydrophilic-lipophilic balance (HLB) of at least about 8. However, it should be understood that emulsifiers and blends of emulsifiers having any other HLB value may also be utilized.

For instance, some nonionic emulsifiers that may be used in the softening composition include, but are not limited to, alkylene oxide esters of fatty acids, alkylene oxide diesters of fatty acids, alkylene oxide ethers of fatty acids, and so forth. Some examples of such alkylene oxide-derived nonionic emulsifiers include, but are not limited to, ceteth-6, ceteth-10, ceteth-12, ceteareth-6, ceteareth-10, ceteareth-12, ceteareth-20, steareth-2, steareth-6, steareth-10, steareth-12, steareth-12, steareth-20, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, glycol stearate, propylene glycol stearate, glycol distearate, glyceryl laurate, glyceryl oleate, and mixtures thereof.

Other suitable nonionic emulsifiers include sugar esters and polyesters, alkoxylated sugar esters and polyesters, polyhydroxy fatty acid amides, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, sorbitan esters, and mixtures thereof. Additional examples of suitable emulsifiers include polyethylene glycol 20 sorbitan monolaurate (Polysorbate 20), polyethylene glycol 5 soy sterol, PPG-2 methyl glucose ether distearate, Polysorbate 80, cetyl phosphate, potassium cetyl phosphate, diethanolamine cetyl phosphate, Polysorbate 60, polyoxyethylene 20 sorbitan trioleate (Polysorbate 85), sorbitan monolaurate, polyoxyethylene 4 lauryl ether sodium stearate, polyglyceryl-4 isostearate, and mixtures thereof. Besides the emulsifiers mentioned above, other types of nonionic emulsifiers, as well as other types of emulsifiers (e.g., cationic, anionic, polymeric, etc.), and blends thereof, may also be utilized. For instance, other suitable emulsifiers that may be utilized in accordance with the present invention are described in U.S. Pat. No. 6,001,377 to SaNogueira, Jr., et al., which is incorporated herein in its entirety by reference thereto for all purposes.

In embodiments where an emulsifier is present, the emulsifier or blend of emulsifiers may be present within the softening composition in an amount between about 0.01% to about 20% by weight of the softening composition, in some embodiments between about 0.01% to about 10% by weight of said softening composition, and in some embodiments, between about 0.01% to about 5% by weight of said softening composition. In one embodiment, steareth-2 (polyoxyethylene-2 stearyl ether) and steareth-20 (polyoxyethylene-20 stearyl ether) and/or steareth-21 (polyoxyethylene-21 stearyl ether) may be used as emulsifiers within the softening composition. For instance, in one embodiment, steareth-2 and steareth-20 and/or steareth-21 may each be present in an amount between about 0.01% to about 20% by weight of the softening composition, in some embodiments between about 0.01% to about 10% by weight of the softening composition, and in some embodiments, between about 0.01% to about 5% by weight of the softening composition.

Various other ingredients may also be incorporated into the aqueous-based softening composition used in the present invention to soften paper-based products. For instance, in some embodiments, an antimicrobial agent (i.e., an additive that is capable of inhibiting the growth-of viruses, bacteria, fungi, and other microbes) may be incorporated into the softening composition to disinfect a user's skin and/or to inhibit the further spread of certain microbes. Typically, an antimicrobial agent utilized in the present invention is biocompatible. For example, some suitable antimicrobial agents that may be used in the softening composition of the present invention include, but are not limited to, chlorhexidine gluconate; parachlorometaxylenol (PCMX); benzylthoneium chloride; chitosan, such as chitosan pyrrolidone carboxylate; 2,4,4′-trichloro-2′-hydroxydiphenyl ether (triclosan), and so forth. Other suitable antimicrobial agents are described in U.S. Pat. No. 5,871,763 to Luu, et al., U.S. Pat. No. 5,334,388 to Hoang, et al., and U.S. Pat. No. 5,686,089 to Mitra, et al., which are incorporated herein in their entirety by reference thereto for all purposes.

The amount of an antimicrobial agent(s) utilized in the softening composition of the present invention may vary. For example, in some embodiments, the amount of the antimicrobial agent(s) may be from about 0.01% to about 10% by weight of the softening composition, in some embodiments from about 0.01% to about 8% by weight of the softening composition, and in some embodiments, between about 0.01% to about 5% by weight of the softening composition.

Furthermore, in some embodiments, the aqueous-based softening composition may also contain one or more preservatives. A preservative may inhibit the growth of certain microbes on the paper-based product before and/or after use. Some suitable preservatives that may be used in the softening composition of the present invention include, but are not limited to, Mackstat H 66 (available from McIntyre Group, Chicago, Ill.), DMDM hydantoin (e.g., Glydant Plus™, Lonza, Inc., Fair Lawn, N.J.), iodopropynyl butylcarbonate, Kathon (Rohm and Hass, Philadelphia, Pa.), methylparaben, propylparaben, 2-bromo-2-nitropropane-1,3-diol, benzoic acid, amidazolidinyl urea, diazolidinyl urea, and so forth. Moreover, in one particular embodiment, a preservative obtained under the name “Phenonip” from NIPA Hardwick may be utilized. Other suitable preservatives includes those sold by Sutton Labs, such as “Germall 115” (amidazolidinyl urea), “Germall II” (diazolidinyl urea), and “Germall Plus” (diazolidinyl urea and iodopropynyl butylcarbonate).

The amount of the preservative(s) utilized-in the softening composition may vary. For example, in some embodiments, the amount of the preservative(s) may be from about 0.01% to about 10% by weight of the softening composition, in some embodiments from about 0.01% to about 8% by weight of the softening composition, and in some embodiments, between about 0.01% to about 5% by weight of the softening composition.

If desired, other ingredients may also be present in the aqueous-based softening composition that is incorporated into paper-based products in the present invention. For instance, some classes of ingredients that may be used include, but are not limited to: antiacne actives (drug products which are used to reduce the number of acne blemishes, acne pimples, blackheads, and whiteheads); antifoaming agents (which reduce the tendency of foaming during processing); antiseptic actives; antioxidants (which increase product integrity); cosmetic astringents (which induce a tightening or tingling sensation on skin); drug astringents (drug products which check oozing, discharge, or bleeding when applied to skin or mucous membrane and which work by coagulating protein); biological additives (which enhance the performance or consumer appeal of the product); colorants; deodorants (which reduce or eliminate unpleasant odor and protect against the formation of malodor on body surfaces); external analgesics (topically applied drugs which have a topical analgesic, anesthetic, or antipruritic effect by depressing cutaneous sensory receptors, or which have a topical counterirritant effect by stimulating cutaneous sensory receptors); film formers (which hold active ingredients on the skin by producing a continuous film on skin upon drying); fragrances (for consumer appeal); opacifiers (which reduce the clarity or transparent appearance of the product); skin exfoliating agents (ingredients that increase the rate of skin cell turnover, such as alpha hydroxy acids and beta hydroxy acids); skin protectants (drug products which protect injured or exposed skin or mucous membrane surfaces from harmful or annoying stimuli); and sunscreens (ingredients that absorb, for instance, at least 85 percent of the light in the UV range at wavelengths from 290 to 320 nanometers, but transmit UV light at wavelengths longer than 320 nanometers. For instance, in one embodiment, aloe vera powder may be utilized in an amount between about 0.0005% to about 0.005% by weight of the softening composition.

The softening composition of the present invention may be incorporated into any of a variety of paper-based products to improve softness. As used herein; the term “paper-based products” may include, but is not limited to, products that are made primarily from a cellulosic fibrous material; products that are made from a cellulosic fibrous material and another component (e.g., a nonwoven web, such as a spunbond or meltblown web); and so forth. Examples of such paper-based products include, but are not limited to, facial tissues, bath tissues, paper towels, napkins, personal care absorbent articles (e.g., diapers, training pants, absorbent underpants, adult incontinence products, feminine hygiene products), wipers, and so forth.

In some embodiments, the paper-based products used in the present invention are single-ply, while in other embodiments the paper-based products are multi-ply. Further, each ply of the paper-based product may be either single-layered or multi-layered. The basis weight of the paper-based products of the present invention may vary dependent on the particular application. In some embodiments, for example, the paper-based product may have a basis weight from about 10 to about 200 grams per square meter (gsm), and in some embodiments, from about 15 to about 100 gsm.

The cellulosic fibrous material in such paper-based products may include cellulosic fibers formed by a variety of pulping processes. Such fibers may include kraft pulp, sulfite pulp, thermomechanical pulp, and so forth. The cellulosic fibers may include softwood fibers, which include, but are not limited to, northern softwood, southern softwood, redwood, red cedar, hemlock, pine (e.g., southern pines), spruce (e.g., black spruce), combinations thereof, and so forth. Exemplary commercially available softwood fibers suitable for the paper-based products of the present invention include those available from Kimberly-Clark Corporation under the trade designation “Longlac 19.”

In some embodiments, the cellulosic fibrous material in the paper-based products may include hardwood fibers, such as eucalyptus, maple, birch, aspen, and so forth. In certain instances, eucalyptus fibers may be particularly desired to increase the softness of the paper-based product to be incorporated with the softening composition described herein. Eucalyptus fibers may also enhance the brightness, increase the opacity, and change the pore structure of the paper-based product to increase its wicking ability.

Moreover, if desired, the cellulosic fibrous material in the paper-based products may include secondary fibers obtained from recycled materials. Such secondary fibers may be derived from various sources, such as newsprint, reclaimed paperboard, and office waste. Processes for recovering and subsequently using such recycled or secondary fibers in paper-based products are disclosed, for example, by U.S. Pat. Nos. 6,372,085; 6,413,363; 6,296,736; and 6,001,218 all to Hsu, et al., which are incorporated herein in their entirety by reference thereto for all purposes.

Further, the cellulosic fibrous material in the paper-based products may include other natural fibers, such as abaca, sabai grass, milkweed floss, pineapple leaf, and so forth. In addition, in some instances, synthetic fibers may also be utilized. Some suitable synthetic fibers may include, but are not limited to, rayon fibers, ethylene vinyl alcohol copolymer fibers, polyolefin fibers, polyesters, and so forth.

As stated above, “paper-based products” include products made primarily from cellulosic fibrous material. Generally, such products may be formed according to a variety of papermaking processes known in the art. For example, a papermaking process suitable for the present invention may utilize through-air-drying, uncreped through-air-drying, creping, double creping, single recreping, double recreping, embossing, wet-pressing, calendering, creped calendaring, air laying, as well as other known steps in forming a paper web. Examples of various papermaking techniques that may be used for making the paper-based products to be treated in the present invention are described in U.S. Pat. Nos. 3,322,617; 3,301,746; 4,158,594; 4,529,480; 4,921,034; and 6,129,815.

More particularly, in certain embodiments, such paper-based products made primarily from cellulosic fibrous material are formed by a non-compressive drying technique, such as uncreped through-drying. In some instances, an uncreped through-dried paper-based product may,have good absorbency and wet-resiliency characteristics. Some examples of uncreped through-drying techniques are disclosed in U.S. Pat. No. 5,048,589 to Cook, et al.; U.S. Pat. No. 5,399,412 to Sudall, et al.; U.S. Pat. No. 5,510,001 to Hermans, et al.; U.S. Pat. No. 5,591,309 to Rugowski, et al.; and U.S. Pat. No. 6,017,417 to Wendt, et al., all. of which are incorporated herein in their entirety by reference thereto for all purposes. For example, uncreped through-drying generally involves the steps of: (1) forming a furnish of cellulosic fibrous material, water, and optionally, other additives; (2) depositing the furnish on a traveling foraminous fabric or wire, thereby forming a fibrous web on top of the traveling foraminous fabric or wire; (3) subjecting the fibrous web to through-drying to remove the water from the fibrous web; and (4) removing the dried fibrous web from the traveling foraminous fabric.

In other embodiments, paper-based products made primarily from cellulosic fibrous material are formed by a creping technique. Some examples of creping techniques are disclosed in U.S. Pat. No. 6,541,099 to Merker, et al., which is incorporated herein in its entirety by reference thereto for all purposes. For example, a creping technique generally involves the steps of: (1) forming a furnish of cellulosic fibrous material, water, and optionally, other additives; (2) depositing the furnish on a traveling foraminous fabric or wire, thereby forming a fibrous web on top of the traveling fabric or wire; (3) drying the fibrous web either compressively or non-compressively to remove the water from the fibrous web; (4) creping one or both sides of the web; and (5) removing the web from the creping apparatus using, for example, a creping blade.

As stated above, in some embodiments of the present invention, the paper-based product is a product made from cellulosic fibrous material and another component (e.g., a nonwoven web, such as a spunbond or meltblown web). For example, the paper-based product may be a wiper formed from a hydroentangled nonwoven fabric. Such a wiper is typically a composite fabric of staple length fibers and a nonwoven web (e.g., a spunbond or meltblown web). Hydroentangling processes and hydroentangled composite webs containing various combinations of different fibers are known in the art. A typical hydroentangling process utilizes high pressure jet streams of water to entangle fibers and/or filaments to form a highly entangled consolidated fibrous structure, e.g., a nonwoven fabric. Hydroentangled nonwoven fabrics of staple length fibers (e.g., pulp fiber) and continuous filaments are disclosed, for example, in U.S. Pat. No. 3,494,821 to Evans; U.S. Pat. No. 4,144,370 to Boulton; U.S. Pat. No. 5,284,703 to Everhart, et al.; and U.S. Pat. No. 6,315,864 to Anderson, et al., which are incorporated herein in their entirety by reference thereto for all purposes.

Another such wiper may be formed from a coform material. The term “coform material” generally refers to composite materials comprising a mixture or stabilized matrix of thermoplastic fibers and a second non-thermoplastic material. As an example, coform materials may be made by a process in which at least one. meltblown die head is arranged near a chute through which other materials are added to a web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Some examples of such coform materials are disclosed in U.S. Pat. No. 4,100,324 to Anderson, et al. and U.S. Pat. No. 5,350,624 to Georger, et al., which are incorporated herein in their entirety by reference thereto for all purposes.

In some embodiments, the aqueous-based softening composition is incorporated into the paper-based product once the product is dry. In general, the softening composition utilized in the present invention may be incorporated into the paper-based product using a variety of methods. For instance, in one embodiment, the softening composition may be applied to the surface of the paper-based product using rotogravure printing, either direct or indirect (offset). Rotogravure printing may sometimes offer better control of the distribution and transfer rate of the softening composition onto the paper-based product. In addition, other application methods, such as flexographic printing, inkjet printing, spraying (either direct or indirect, e.g., WEKO spraying), hot melt adhesive spraying (e.g., Nordson), blade, saturant, coating, droplet throw, and foam applications, may be used.

The softening composition of the present invention may, in some embodiments, be applied to or incorporated into one or both outer surfaces of the paper-based product. For instance, when utilizing a multi-ply paper-based product, the softening composition may be applied after the plies are brought together or prior to bringing the plies together. The individual plies may be layered or blended (homogeneous), creped or uncreped, through-dried or wet-pressed, and so forth.

The softening composition may be incorporated into the paper-based product such that the add-on level is relatively low. As used herein, the term “add-on level” refers to the percentage of softening composition present on the paper-based product after the softening composition has been incorporated into the paper-based product. The add-on level is calculated by subtracting the pre-treatment weight of the paper-based product from the post-treatment weight of the paper-based product and dividing this difference by the pre-treatment weight of the paper-based product. This quotient is then multiplied by 100 to obtain a percentage, used as the add-on-level.

Relatively low add-on levels of the softening composition may be desired in the present invention so that the treated paper-based products substantially retain their absorbency. Further, the silicone components present in the softening composition, such as silicone glycols and silicone quaternary ammonium compounds, may be expensive; therefore, low add-on levels of softening composition reduce the overall cost of the chemically treated, softness-improved paper-based products. Thus, the softening composition may be incorporated into the paper-based product such that the add-on level is between about 0.1% and about 10% by weight of the paper-based product, in some embodiments between about 0.5% and about 10% by weight of the paper-based product, and in other embodiments between about 0.5% and about 5% by weight of the paper-based product.

The aqueous-based softening composition applied to paper-based products in accordance with the present invention includes a silicone glycol, a silicone quaternary ammonium compound, an emollient, water, and other optional ingredients (a fatty alcohol, a skin conditioning agent, an emulsifier, antimicrobial agents, preservatives, fragrances, and so forth). As mentioned above, the present inventor has unexpectedly discovered that chemically treating paper-based products with the softening composition described herein produces paper-based products having synergistic levels of improved softness. Moreover, paper-based products treated in accordance with the present invention exhibit improved softness without exhibiting significant decreases in absorbency and/or paper strength.

The present invention may be better understood with reference to the following example.

### EXAMPLE

Various chemical treatments were applied to two-ply base sheets containing either 100% recycled cellulosic fibers or a mixture of recycled cellulosic fibers and eucalyptus hardwood fibers. The base sheets were all made using a creping technique, wherein an aqueous furnish of cellulosic fibers (either 100% recycled fibers or a mixture of recycled fibers and eucalyptus hardwood fibers) was provided and deposited on a traveling foraminous wire, thereby forming a fibrous web on top of the traveling wire. The fibrous web was dried and creped using a Yankee dryer, and the resulting two-ply base sheets had a basis weight range of from about 25 to about 27 grams per square meter (gsm).

As summarized in Table 1 below, Samples 1-4 were used as “control” base sheets, which underwent no chemical treatment after they were formed. Samples 1-3 were base sheets made from 100% recycled fibers, while Sample 4 was a base sheet made from 70% recycled fibers and 30% eucalyptus hardwood fibers.

Samples 5-22 were base sheets that were applied with a variety of chemical treatments, summarized in Table 1 below. Samples 5-22 were base sheets made from 100% recycled fibers. Samples 5-22 were all chemically treated using the same laboratory hand spraying technique. Specifically, the treatment solution for each base sheet, summarized in Table 1 below, was first loaded into a pressurized hand sprayer connected to a cylinder of compressed air. This pressurized hand sprayer was then used to spray a mist of the treatment solution into the air space above the first side of each base sheet. The mist of the treatment solution settled on the surface of the first side, and the spraying continued until about 50% of the treatment solution originally loaded into the sprayer was used. Subsequently, each base sheet was turned over, and the remaining 50% of the treatment solution was sprayed, as a mist, into the air space above the second side of the base sheet. This mist of the treatment solution settled on the surface of the second side of the base sheet.

Once both sides of each base sheet were applied with the treatment solution of choice, the add-on level of chemical treatment was determined by subtracting the pre-treatment weight of the base sheet from its post-treatment weight before drying and dividing this difference by the pre-treatment weight of the base sheet. This quotient was then multiplied by 100 to obtain a percentage, noted in Table 1 below as the “add-on level.” After the add-on level of chemical treatment was determined, each treated base sheet was then air-dried. The chemical treatments applied to the base sheets are briefly described in Table 1 below:

TABLE 1
Chemical Treatments Applied to Samples 1-22

| | | Treatment |
|---|---|---|
| | | Add-on |
| Sample No. | Chemical Treatment | Level |
| 1 | None (100% Recycled Fibers) | — |
| 2 | None (100% Recycled Fibers) | — |
| 3 | None (100% Recycled Fibers) | — |
| 4 | None (70% Recycled Fibers, 30% Eucalyptus Fibers) | — |
| 5 | Quat-based Debonder (Rewoquat V3611) | 0.2% |
| 6 | Quat-based Debonder (Rewoquat V3611) | 0.4% |
| 7 | Quat-based Debonder (Quaker Quasoft) | 0.2% |
| 8 | Quat-based Debonder (Quaker Quasoft) | 0.4% |
| 9 | Quat-based Debonder (Quaker Quasoft) | 0.2% |
| 10 | Quat-based Debonder (Quaker Quasoft) | 0.4% |
| 11 | Quat-based Debonder (C.Y. Witco Y14344) | 0.3% |
| 12 | Quat-based Debonder (C.Y. Witco Y14344) | 0.5% |
| 13 | Silicone glycol/silicone diquat + Mackernium 601 DES | 1.0% |
| 14 | Silicone glycol/silicone diquat + Mackernium 601 DES | 2.0% |
| 15 | Silicone glycol/silicone diquat + Mackernium 601 DES + Lotion | 1.0% |
| 16 | Silicone glycol/silicone diquat + Mackernium 601 DES + Lotion | 2.0% |
| 17 | Mackernium 601 DES + Lotion | 3.0% |
| 18 | Mackernium 601 DES + Lotion | 4.0% |
| 19 | Silicone glycol/silicone diquat + Lotion | 2.0% |
| 20 | Silicone glycol/silicone diquat + Lotion | 3.0% |
| 21 | Silicone glycol/silicone diquat + Lotion | 2.0% |
| 22 | Silicone glycol/silicone diquat + Lotion | 3.0% |

Samples 5-12 were base sheets that had been treated with aqueous solutions of various quat-based debonders. Samples 5 and 6 underwent treatment with a quat-based debonder commercially available as Rewoquat V3611 (Goldschmidt Chemical Corp., Dublin, Ohio), and the add-on levels for Samples 5 and 6 were determined to be 0.2% and 0.4%, respectively. Samples 7-10 underwent treatment with a quat-based debonder commercially available as Quaker Quasoft (Quaker Corp., Conshohocken, Pa.), and the add-on levels for Samples 7-10 were determined to be 0.2% and 0.4%, as listed in Table 1 above. Samples 11-12 underwent treatment with a quat-based debonder commercially available as C.Y. Witco Y14344 (Witco Corp., Tarrytown, N.Y.), and the add-on levels for Samples 11 and 12 were determined to be 0.3% and 0.5%, respectively.

Samples 13-22 were base sheets that had been treated with various aqueous-based softening compositions. For all of Samples 13-22, the respective softening compositions were formed simply by adding together the listed ingredients and mixing using mild agitation. Specifically, Samples 13-14 were base sheets that had been treated with an aqueous-based softening composition containing 14% by chemical weight of Glensil WS 100 and 14% by chemical weight of Mackernium 601 DES. Glensil WS 100 is an organo-modified silicone commercially available from Glenn Corporation, St. Paul, Minn. More particularly, Glensil WS 100 is a clear liquid having a pH of about 5.84 and contains at least one silicone glycol and at least one silicone diquaternary ammonium compound. Mackernium 601 DES contains behenyl quat and polyethylene glycol (PEG) 400 and is commercially available from McIntyre Group Ltd. in University Park, Ill. The add-on levels of softening composition for Samples 13 and 14 were determined to be 1% and 2%, respectively.

Samples 15 and 16 were base sheets that had been treated with an aqueous-based softening composition containing: 7% by chemical weight of Glensil WS 100, the organo-modified silicone described above; 7% by chemical weight of Mackernium 601 DES, also described above; 50% by chemical weight of a lotion commercially available as KimCare™ Moisturizing Hand & Body Lotion from Kimberly-Clark; and 36% water. The add-on levels of softening composition for Samples 15 and 16 were determined to be 1% and 2%, respectively.

KimCare™ Moisturizing Hand & Body Lotion includes water, an emollient, and various other ingredients, which are listed in Table 2 below:

TABLE 2
Composition of KimCare ™ Moisturizing
Hand & Body Lotion

| Ingredient | Weight % of Lotion |
|---|---|
| Water | 85.288 |
| C₁₂-C₁₅ alkyl benzoate | 2.000 |
| Cetearyl alcohol | 3.500 |
| Glycerin | 2.000 |
| Caprylic/capric stearate triglyceride | 2.000 |
| Steareth-2 | 1.200 |
| Dimethicone | 1.000 |
| Steareth-20 | 0.900 |
| Chlorhexidine gluconate (20%) | 1.250 |
| Stearamidopropyl PG-dimonium | 0.600 |
| chloride phosphate and cetyl alcohol | |
| Methylparaben | 0.200 |
| Fragrance | 0.060 |
| Tocopheryl acetate (Vitamin E) | 0.001 |
| Panthenol (Pro-Vitamin B5) | 0.001 |

More particularly, in the above formulation of the KimCare™ lotion, C₁₂-C₁₅ alkyl benzoate is the emollient, commercially available as Finsolv TN from Finetex, Inc. in Elmwood Park, N.J. Additionally, cetearyl alcohol is a specific fatty alcohol used and is commercially available as Lipocol CS-50 from Lipo Chemicals, Inc. in Paterson, N.J. The steareth-2 and steareth-20 emulsifiers used in the KimCare™ lotion are commercially available as Lipocol S-2 and Lipocol S-20, respectively, from Lipo Chemicals, Inc. in Paterson, N.J. Skin conditioning agents in the above lotion include glycerin, caprylic/capric stearate triglyceride (commercially available as Softisan 378 from CONDEA Vista Corp. in Houston, Tex.), dimethicone, and stearamidopropyl PG-dimonium chloride phosphate and cetyl alcohol (commercially available as Phospholipid SV from Mona Industries in Paterson, N.J.). An antimicrobial agent used in the above lotion is chlorhexidine gluconate, and methylparaben is included as a preservative. Further the fragrance used in the lotion is commercially available as Arylessene #AA01164.

Returning to the chemically treated samples listed in Table 1 above, Samples 17 and 18 were base sheets that had been treated with an aqueous-based softening composition containing: 7% by chemical weight of Mackernium 601 DES, described above; 46.5% by chemical weight of the KimCare™ lotion, described above; and 46.5% water. The add-on levels of softening composition for Samples 17 and 18 were determined to be 3% and 4%, respectively.

Furthermore, Samples 19-22 were base sheets that had been treated with an aqueous-based softening composition containing: 7% by chemical weight of Glensil WS 100, the organo-modified silicone described above; 46.5% by chemical weight of the KimCare™ lotion, described above; and 46.5% by weight water. The add-on levels of softening composition for Samples 19-22 were determined to be 2% and 3% as listed in Table 1 above.

Once the twenty-two base sheets were treated with the chemical treatments discussed above, the chemically-treated base sheets were then tested for several tactile properties to determine whether such properties were improved as a result of the various chemical treatments. The following tests were utilized:

Geometric Mean Tensile Strength Testing:

The Geometric Mean Tensile (GMT) strength test results are expressed as grams-force per 3 inches of sample width. GMT is computed from the peak load values of the MD (machine direction) and CD (cross-machine direction) tensile curves, which are obtained under laboratory conditions of 23.0±1.0 degrees Celsius, 50.0±2.0 percent relative humidity, and after the sheet has equilibrated to the testing conditions for a period of not less than four hours. Testing is done on a tensile testing machine maintaining a constant rate of elongation, and the width of each specimen tested was 3 inches. The “jaw span” or the distance between the jaws, sometimes referred to as gauge length, is 2.0 inches (50.8 mm). Crosshead speed is 10 inches per minute (254 mm/min.) A load cell or full-scale load is chosen so that all peak load results fall between 10 and 90 percent of the full-scale load.

In particular, the results described herein were produced on an Instron 1122 tensile frame connected to a Sintech data acquisition and control system utilizing IMAP software running on a “486 Class” personal computer. This data system records at least 20 load and elongation points per second. A total of 5 specimens per sample are tested with the sample mean being used as the reported tensile value. The geometric mean tensile is calculated from the following equation:
GMT=(MD Tensile*CD Tensile)^{1/2}
The GMT values for the above-described Samples 1-22 are reported in Table 3 below.
Sensory Profile Panel Testing:

Silkiness, Fuzziness, Grittiness, and Stiffness values were obtained through a Sensory Profile Panel testing method. A group of 15-20 trained panelists were given the series of treated paper products, one sample at a time. Samples were in the form of two continuous sheets for each sample. For each sample, the panelists rated the tissue for fuzziness, grittiness, and stiffness on a scale of 1 (low) to 16 (high) in a sequential fashion.

Specifically, for the Silkiness test, the panelists determined how smooth or silky each sample was to the touch. The panelists placed each sample flat on a table and, using the full length of their index and middle fingers, glided their fingers across the entire surface of each sample. The panelists tested for silkiness in the long direction of each sample.

For the Fuzziness test, the panelists determined how much pile or fuzz was on the surface of each sample. The panelists placed each sample flat on a table and, using the pads of their index and middle fingers, moved in quarter-sized circular motions, lightly across several areas of each sample.

During the Grittiness testing, the panelists determined the general abrasiveness or roughness of the sample, paying attention to the amount of sharp, prickly particles or fibers felt on the surface of each sample. The panelists placed each sample flat on a table and, using the full length of their index and middle fingers, glided their fingers across the entire surface of each sample. The panelists tested for grittiness in the long direction of each sample.

For the Stiffness testing, the panelists determined the amount of pointed, ridged, or sharp folds or peaks in each sample by gently moving the entire gathered sample around in the palm of the hand. The results reported in Table 3 below are an average of panel rankings for Samples 1-22.

TABLE 3
Tactile Properties of Base Sheet Samples 1-22

| Sample | GMT | | | | |
|---|---|---|---|---|---|
| No. | (g/3″) | Silky | Fuzzy | Gritty | Stiffness |
| 1 | 652 | 6.58 | 3.30 | 3.12 | 6.28 |
| 2 | 655 | 6.82 | 3.60 | 3.42 | 6.38 |
| 3 | 690 | 6.66 | 3.54 | 3.02 | 6.22 |
| 4 | 629 | 6.86 | 3.52 | 3.18 | 5.64 |
| 5 | 579 | 6.62 | 3.62 | 3.00 | 6.32 |
| 6 | 648 | 6.66 | 3.88 | 2.98 | 5.74 |
| 7 | 604 | 7.06 | 3.88 | 3.02 | 6.38 |
| 8 | 614 | 6.88 | 3.56 | 2.80 | 5.78 |
| 9 | 520 | 7.00 | 3.50 | 2.98 | 5.90 |
| 10 | 584 | 6.76 | 3.50 | 2.92 | 5.92 |
| 11 | 651 | 6.74 | 3.46 | 3.20 | 5.86 |
| 12 | 550 | 6.92 | 3.26 | 2.76 | 5.78 |
| 13 | 602 | 6.68 | 3.86 | 3.12 | 5.96 |
| 14 | 534 | 6.96 | 3.62 | 2.62 | 5.58 |
| 15 | 631 | 6.84 | 3.80 | 2.96 | 6.16 |
| 16 | 492 | 6.98 | 3.60 | 3.24 | 5.90 |
| 17 | 584 | 6.78 | 3.48 | 3.20 | 6.26 |
| 18 | 571 | 6.62 | 3.7 | 3.20 | 6.18 |
| 19 | 544 | 7.08 | 3.62 | 3.04 | 5.92 |
| 20 | 524 | 7.30 | 3.82 | 2.66 | 5.68 |
| 21 | 542 | 7.24 | 3.78 | 2.58 | 5.46 |
| 22 | 515 | 7.02 | 3.52 | 3.10 | 5.32 |

The results reported in Table 3 above show that Samples 19-22, base sheets which were treated with an aqueous-based softening composition that included a silicone glycol/silicone diquat-containing composition, water, and an emollient-containing lotion, exhibited low levels of stiffness and high levels of silkiness, illustrating improved softness over the control base sheets used as Samples 1-4. Further, Samples 15 and 16, base sheets which were treated with an aqueous-based softening composition that included the silicone glycol/silicone diquat-containing composition, water, the emollient-containing lotion, and Mackernium 601 DES, showed increased silkiness and decreased stiffness compared to control Samples 1-4, though not as marked of an increase in silkiness or a decrease in stiffness as Samples 19-22 showed when compared to control Samples 1-4. The above Example shows that treating a paper-based product with an aqueous-based softening composition according to the present invention unexpectedly leads to treated paper-based products having improved softness where the strength of the treated paper-based products has not been significantly decreased or sacrificed.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A soft paper-based product comprising a cellulosic fibrous material, wherein an aqueous-based softening composition is incorporated into the paper-based product at an add-on level of between about 0.1% to about 10% by weight of the paper-based product, said softening composition comprising a silicone glycol in an amount between about 0.01% to about 20% by weight of said softening composition, a silicone quaternary ammonium compound in an amount between about 0.01% to about 20% by weight of said softening composition, an emollient in an amount between about 0.01% to about 20% by weight of said softening composition, and water in an amount greater than about 40% by weight of said softening composition.

2. The product of claim 1, wherein said silicone glycol has the general formula: 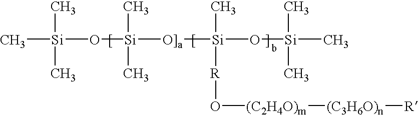
wherein a≧1 and b≧1;
wherein R is selected from C₁-C₆ alkyls and C₁-C₆ hydroxyalkyls;
wherein R′ is selected from hydrogen, C₁-C₆ alkyls, C₁-C₆ hydroxyalkyls, C₁-C₆ acyls, and C₁-C₆ acetates;
wherein m is between about 1 to about 500; and
wherein n is between about 1 to about 300.

3. The product of claim 1, wherein said silicone quaternary ammonium compound has the general formula: 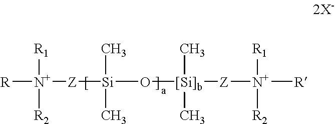
wherein a≧1 and b≧1;
wherein R₁ is selected from C₁-C₆ alkyls and C₁-C₆ hydroxyalkyls;
wherein R₂ is selected from C₁-C₆ alkyls and C₁-C₆ hydroxyalkyls;
wherein R is selected from C₈-C₂₄ aliphatic hydrocarbons;
wherein R′ is selected from C₈-C₂₄ aliphatic hydrocarbons;
wherein Z is selected from the group consisting of: —(CH₂)ⱼ—CHOH—CH₂—O—(CH₂)ₖ—, wherein j≧1 and k≧1; CH₂—CH₂—CH₂—O—(CH₂)₃—; alkyls; and alkyl esters; and
wherein X is an ion.

4. The product of claim 1, wherein said water comprises greater than about 75% by weight of said softening composition.

5. The product of claim 1, wherein said emollient comprises between about 0.01% to about 10% by weight of said softening composition.

6. The product of claim 1, wherein said emollient includes a linear primary alkyl ester of benzoic acid.

7. The product of claim 6, wherein said linear primary alkyl ester of benzoic acid is C₁₂-C₁₅ alkyl benzoate.

8. The product of claim 1, wherein said softening composition further comprises a fatty alcohol in an amount between about 0.01% to about 20% by weight of said softening composition.

9. The product of claim 8, wherein said fatty alcohol is selected from the group consisting of cetyl alcohol, stearyl alcohol, cetearyl alcohol, arachidyl alcohol, behenyl alcohol, and combinations thereof.

10. The product of claim 1, wherein said softening composition further comprises an emulsifier in an amount between about 0.01% to about 20% by weight of said softening composition.

11. The product of claim 10, wherein said emulsifier includes a polyoxyethylene stearyl ether.

12. The product of claim 1, wherein said softening composition further comprises a skin conditioning agent in an amount between about 0.01% to about 20% by weight of said softening composition.

13. The product of claim 12, wherein said skin conditioning agent includes a humectant.

14. The product of claim 13, wherein said humectant includes glycerin.

15. The product of claim 1, wherein said add-on level of said softening composition is between about 0.5% to about 10% by weight of said paper-based product.

16. The product of claim 1, wherein said softening composition further comprises an antimicrobial agent, a preservative, or combinations thereof.

17. The product of claim 1, wherein said paper-based product has a basis weight between about 10 to about 200 grams per square meter.

18. The product of claim 1, wherein said paper-based product has a basis weight between about 15 to about 100 grams per square meter.

19. A method for forming a soft paper product comprising:
forming a web from at least one furnish containing cellulosic fibrous material and water; and
treating said web with an aqueous-based softening composition such that the add-on level of said softening composition is between about 0.1% to about 10% of said paper product,
wherein said softening composition comprises a silicone glycol in an amount between about 0.01% to about 20% by weight of said softening composition, a silicone quaternary ammonium compound in an amount between about 0.01% to about 20% by weight of said softening composition, an emollient in an amount between about 0.01% to about 20% by weight of said softening composition, and water in an amount greater than about 40% by weight of said softening composition.

20. The method of claim 19, further comprising drying said web.

21. The method of claim 20, wherein said web is through-dried.

22. The method of claim 20, wherein said web is treated with said softening composition after said web is dried.

23. The method of claim 19, further comprising creping said web.

24. The method of claim 19, wherein said silicone glycol has the general formula: 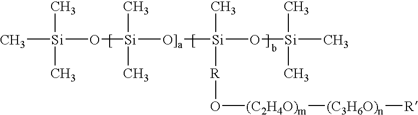
wherein a≧1 and b≧1;
wherein R is selected from C₁-C₆ alkyls and C₁-C₆ hydroxyalkyls;
wherein R′ is selected from hydrogen, C₁-C₆ alkyls, C₁-C₆ hydroxyalkyls, C₁-C₆ acyls, and C₁-C₆ acetates;
wherein m is between about 1 to about 500; and
wherein n is between about 1 to about 300.

25. The method of claim 19, wherein said silicone quaternary ammonium compound has the general formula: 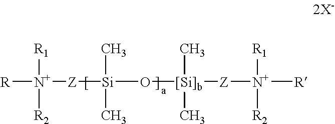
wherein a≧1 and b≧1;
wherein R₁ is selected from C₁-C₆ alkyls and C₁-C₆ hydroxyalkyls;
wherein R₂ is selected from C₁-C₆ alkyls and C₁-C₆ hydroxyalkyls;
wherein R is selected from C₈-C₂₄ aliphatic hydrocarbons;
wherein R′ is selected from C₈-C₂₄ aliphatic hydrocarbons;
wherein Z is selected from the group consisting of: —(CH₂)ⱼ—CHOH—CH₂—O—(CH₂)ₖ—, wherein j≧1 and k≧1; CH₂—CH₂—CH₂O—(CH₂)₃—; alkyls; and alkyl esters; and
wherein X is an ion.

26. The method of claim 19, wherein said water comprises greater than about 75% by weight of said softening composition.

27. The method of claim 19, wherein said emollient comprises between about 0.01% to about 10% by weight of said softening composition.

28. The method of claim 19, wherein said emollient includes a linear primary alkyl ester of benzoic acid.

29. The method of claim 28, wherein said linear primary alkyl ester of benzoic acid is C₁₂-C₁₅ alkyl benzoate.

30. The method of claim 19, wherein said add-on level of said softening composition is between about 0.5% to about 10% by weight of said paper product.

31. The method of claim 19, wherein said paper product has a basis weight between about 10 to about 200 grams per square meter.

32. The method of claim 19, wherein said paper product has a basis weight between about 15 to about 100 grams per square meter.

33. An aqueous-based softening composition comprising:
a silicone glycol in an amount between about 0.01% to about 20% by weight of said softening composition;
a silicone quaternary ammonium compound in an amount between about 0.01% to about 20% by weight of said softening composition;
an emollient in an amount between about 0.01% to about 20% by weight of said softening composition; and
water in an amount greater than about 40% by weight of said softening composition.

34. The softening composition of claim 33, wherein said silicone glycol has the general formula: 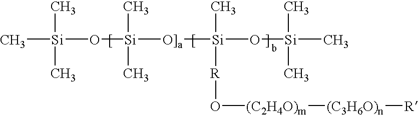
wherein a≧1 and b≧1;
wherein R is selected from C₁-C₆ alkyls and C₁-C₆ hydroxyalkyls;
wherein R′ is selected from hydrogen, C₁-C₆ alkyls, C₁-C₆ hydroxyalkyls, C₁-C₆ acyls, and C₁-C₆ acetates;
wherein m is between about 1 to about 500; and
wherein n is between about 1 to about 300.

35. The softening composition of claim 33, wherein said silicone quaternary ammonium compound has the general formula: 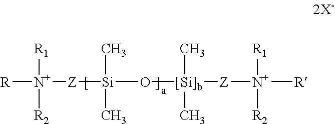
wherein a≧1 and b≧1;
wherein R₁ is selected from C₁-C₆ alkyls and C₁-C₆ hydroxyalkyls;
wherein R₂ is selected from C₁-C₆ alkyls and C₁-C₆ hydroxyalkyls;
wherein R is selected from C₈-C₂₄ aliphatic hydrocarbons;
wherein R′ is selected from C₈-C₂₄ aliphatic hydrocarbons;
wherein Z is selected from the group consisting of: —(CH₂)ⱼ—CHOH—CH₂—O—(CH₂)ₖ—, wherein j≧1 and k≧1; CH₂CH₂—CH₂—O—(CH₂)₃—; alkyls; and alkyl esters; and
wherein X is an ion.

36. The softening composition of claim 33, wherein said water comprises greater than about 75% by weight of said softening composition.

37. The softening composition of claim 33, wherein said emollient comprises between about 0.01% to about 10% by weight of said softening composition.

38. The softening composition of claim 33, wherein said emollient includes a linear primary alkyl ester of benzoic acid.

39. The softening composition of claim 38, wherein said linear primary alkyl ester of benzoic acid is C₁₂-C₁₅ alkyl benzoate.

40. The softening composition of claim 33, further comprising a fatty alcohol in an amount between about 0.01% to about 20% by weight of said softening composition.

41. The softening composition of claim 40, wherein said fatty alcohol is selected from the group consisting of cetyl alcohol, stearyl alcohol, cetearyl alcohol, arachidyl alcohol, behenyl alcohol, and combinations thereof.

42. The softening composition of claim 33, wherein said softening composition is an oil-in-water emulsion.

43. The softening composition of claim 33, further comprising an emulsifier in an amount between about 0.01% to about 20% by weight of said softening composition.

44. The softening composition of claim 43, wherein said emulsifier includes a polyoxyethylene stearyl ether.

45. The softening composition of claim 33, further comprising a skin conditioning agent in an amount between about 0.01% to about 20% by weight of said softening composition.

46. The softening composition of claim 45, wherein said skin conditioning agent includes a humectant.

47. The softening composition of claim 46, wherein said humectant includes glycerin.

48. The softening composition of claim 33, further comprising an antimicrobial agent, a preservative, or combinations thereof.
